(19) United States
(12) Patent Application Publication
(10) Pub. No.: US 2007/0184015 A1
(43) Pub. Date: Aug. 9, 2007

(54) Novel PEGylation agent

(76) Inventor: SoonKap Hahn, San Clemente, CA (US)

Correspondence Address: MICHAEL E. KLICPERA, PO BOX 573, LA JOLLA, CA, 92038-0573, US

(21) Appl. No.: 11/346,878

(22) Filed: Feb. 3, 2006

(51) Int. Cl. A61K 38/04; C08L 89/00

(52) U.S. Cl. 424/78.27; 514/19; 525/54.1

(57) ABSTRACT

To address the issue of degradation by enzymatic reactions to proteins and peptides, polyethylene glycol (PEGylation) of the proteins and peptides has been established. PEGylated proteins and peptides have increased plasma half-lives and reduced immunogenicity. To further improve and extend the plasma half-life of desired protein or peptide therapeutics, a novel branched molecule of PEG possessing three PEGs with a single point of attachment is designed in this invention disclosure.

### FIELD OF THE INVENTION

The present invention relates generally to the field of polyethylene glycol (PEG) and proteins or peptides, in particular, to compositions and methods for the fabrication of biodegradable PEGylated proteins and peptides.

### BACKGROUND OF THE INVENTION

Without limiting the scope of the invention, for purposes of the ensuing discussion, description and claims of the present invention, the terms “protein”, “polypeptide” and “peptide” may be used interchangeably, although it will be appreciated by those skilled in the art that biological distinctions may be drawn between them and, as such distinctions do not affect the operation of the present invention, such distinctions as may be drawn are contemplated by the scope of the present invention.

The advent of recombinant DNA and protein technology makes possible the production of significant quantities of both DNA and proteins for use in the clinical setting. The appeal of recombinant therapeutics is enhanced by delivery systems that provide controlled pharmacokinetics of the desired therapy. Paramount to the development of using DNA and protein technology is to assurance that the biological activity of the material is preserved throughout delivery and release within a patient to allow the therapeutic activity to treat the targeted condition successfully.

A variety of approaches have been developed to permit controlled, sustained release of a biologically active agent into a subject so enzymatic degradation is diminished. Examples of controlled release systems include the polymeric compositions described in U.S. Pat. Nos. 4,938,763; 5,278,201 and 5,278,202. The compositions described in these patents are administered to the body of a subject in a flowable state. Once in the body, the composition coagulates or cures to form a solid implant.

One polymeric composition includes a thermoplastic polymer or copolymer, an organic solvent and a biologically active agent. The thermoplastic polymer is biocompatible, biodegradable and substantially insoluble in aqueous body or tissue fluids. The organic solvent is also biocompatible and miscible to dispersible in aqueous body or tissue fluids. The polymeric composition is flowable and can be introduced into the body using a syringe, for example. When the polymeric composition comes into contact with an aqueous medium such as body or tissue fluid, the solvent dissipates or diffuses into the aqueous medium. Concurrently, the substantially insoluble thermoplastic polymer precipitates or coagulates to form a solid implant. As the thermoplastic polymer precipitates or coagulates to form the solid matrix, the active agent is trapped or encapsulated throughout the polymeric matrix. The biologically active agent is then released by dissolution or diffusion through the polymeric matrix and/or the biologically active agent is released as the matrix biodegrades.

However, the formation of the solid matrix from the flowable delivery system is not instantaneous. Typically the process can occur over a period of minutes to several hours. During this period, the rate of diffusion of the biologically active agent from the coagulating polymeric composition may be much more rapid than the rate of release that occurs from the subsequently formed solid matrix.

Another method for shield against enzymatic degradation is to utilize biodegradable microspheres. Methods are known to encapsulate various proteins within biodegradable microspheres. In particular, polypeptides have been incorporated into Poly (DL-lactide- co-glycolide) (PLGA) microspheres with varying degrees of success. PLGA is a polymer that has been used for many years as a biodegradable suture material. PLGA is biocompatible and degrades by hydrolytic cleavage into nontoxic molecules that are easily eliminated from the body (namely, lactic acid and glycolic acid). In addition to polypeptide micro-encapsulation, sustained delivery of polypeptides is also possible through the use of biodegradable microspheres. A non-exhaustive list of such polypeptides includes nerve growth factor, alpha, beta and gamma interferon, growth hormone, insulin erythropoietin, transforming growth factor beta, epidermal growth factor interleukin-2, basic fibroblast growth factor and vascular endothelial growth factor. PLGA has been described, therefore, as a desirable polymer for use as a drug delivery system. Preserving the biological activity of the microencapsulated polypeptide, however, has proven to be problematic and has retarded the development of microencapsulation drug delivery.

The double-emulsion technique is the most commonly reported method for manufacturing microspheres. According to this technique, protein dissolved in an aqueous solution is then emulsified in an organic solvent containing the dissolved PLGA. The aqueous-organic emulsion is then further emulsified in an aqueous alcohol phase to create an aqueous-organic-aqueous double emulsion. The alcohol phase extracts the organic solvent away from the PLGA in approximately one hour, leaving the protein entrapped in discrete droplets within solid microspheres. The process of emulsifying the aqueous protein solution in the organic solvent, however, can easily denature the protein.

Protein may be encapsulated into microspheres by known methods either in solution or as a solid. The incorporation of solid protein into a microsphere has previously been accomplished by the atomization-freeze (AF) process. The AF process requires the use of an ultrasonic atomizer with a custom designed spray nozzle. A description of the atomization-freeze technique is found in Putney S. D., Burke P. A., “Improving Protein Therapeutics with Sustained-release Formulations,” Nat. Biotechnol 1998; 16:153-157. Another method for the encapsulation of solid proteins is described in Cao X., Schoichiet M. S., “Delivering Neuroactive Molecules from Biodegradable Microspheres for Application in Central Nervous System Disorders,” Biomaterials 1999; 20:329-339. Briefly, Cao and Schoichet dispersed ovalbumin powder in a solution of PLGA in chloroform using a Polytron homogenizer. The protein-polymer dispersion was added to an aqueous solution of 1% polyvinyl alcohol (PVA) and homogenized again to form an emulsion. The emulsion was added to more PVA solution and stirred continuous to evaporate the organic solvent. The microspheres were centrifuged, washed and freeze dried.

One problem with the use of microspheres is the use of a single polymer species such as PLGA alone, or a PLGA/poly(eta-caprolactine) to fabricate the microspheres. Certain advantages in the release kinetics of the microspheres may be achieved by using a mixture of polymers rather than a single polymer species to fabricate the microspheres. Release kinetics are determined, in part, by the amount of bioactive substances loaded, the polymer or polymers used and the conditions of manufacture. The particle size of the microspheres is determined to a large extent by the manufacturing conditions such as polymer viscosity and the method of physical shearing used to produce the microspheres. Methods of shearing include but are not limited to homogenization with a tissue homogenizer or blender, ultrasound sonication, or vibrating with the use of a VORTEX® mixer. Smaller particles have a faster rate of degradation due to the increased ratio of surface area to volume. Thus, for microspheres composed of PLGA alone, the release of protein is generally regulated only by the physical erosion of the polymer, particularly where the protein/polymer ratio of the microsphere is low.

Another problem with the use of microspheres method is the use of a tissue homogenizer to form the polymer emulsion. In terms of commercial scale-up of microsphere production, a tissue homogenizer is impractical. Homogenizers have parts such as blades, rotors and containers that require cleaning and sterilization between each batch. The care and maintenance of homogenizers renders them problematic for the large scale production of pharmaceutical microspheres.

Additionally, the use of single straight-chain molecule of polyethylene glycol (PEG) is attached at a single point to a peptide or protein has been employed with limited success. Later, dual-branched PEG (two PEGs with a single point of attachment, was developed to provide the additional steric bulk providing a better yet still limited polymeric shield against enzymatic degradation.

### SUMMARY OF THE INVENTION

Proteins and peptides are an important class of therapeutics. However, when such proteins and peptides are administered therapeutically in their natural form, proteolytic enzymes can degrade them very quickly. Furthermore, such administered proteins and peptides may also elicit an immunogenic response. The short plasma half-life caused by rapid degradation requires frequent dosing, resulting in reduced compliance by patients for their needed treatments.

To address this problem, the derivatization of proteins and peptides with polyethylene glycol (PEGylation) has been established. PEGylated proteins and peptides have increased plasma half-lives and reduced immunogenicity[references needed here?]. Traditionally a single straight-chain molecule of polyethylene glycol (PEG) was attached at a single point to a peptide or protein providing some protection from enzymatic degradation. Subsequently, branched PEG (two PEGs with a single point of attachment) was developed to provide the additional steric bulk, thus providing a better polymeric shield against enzymatic degradation[reference?].

To further improve and extend the plasma half-life and reduce immunogenic properties of desired protein, peptide or other therapeutic agents, a novel branched molecule of PEG possessing three PEGs with a single point of attachment is designed as the present invention.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1 demonstrates molecules traditionally used for PEGylation: Single straight chain molecule (I) and branched PEG (two PEGs with a single point of attachment, II)

FIG. 2 demonstrates a molecule having three amine groups and one carboxyl group used as the seed molecule for manufacturing the present invention.,

FIG. 3 demonstrates a molecule which demonstrates that the three amine groups are coupled with an active monomethoxy-polyethylene glycol (mPEG) molecule (III).

FIG. 4 demonstrates the present invention with a branched design having three PEGs with a single activated carboxyl group.

FIG. 5 demonstrates the present invention with a branched design having three PEGs with the single activated carboxyl group coupled to a medicament.

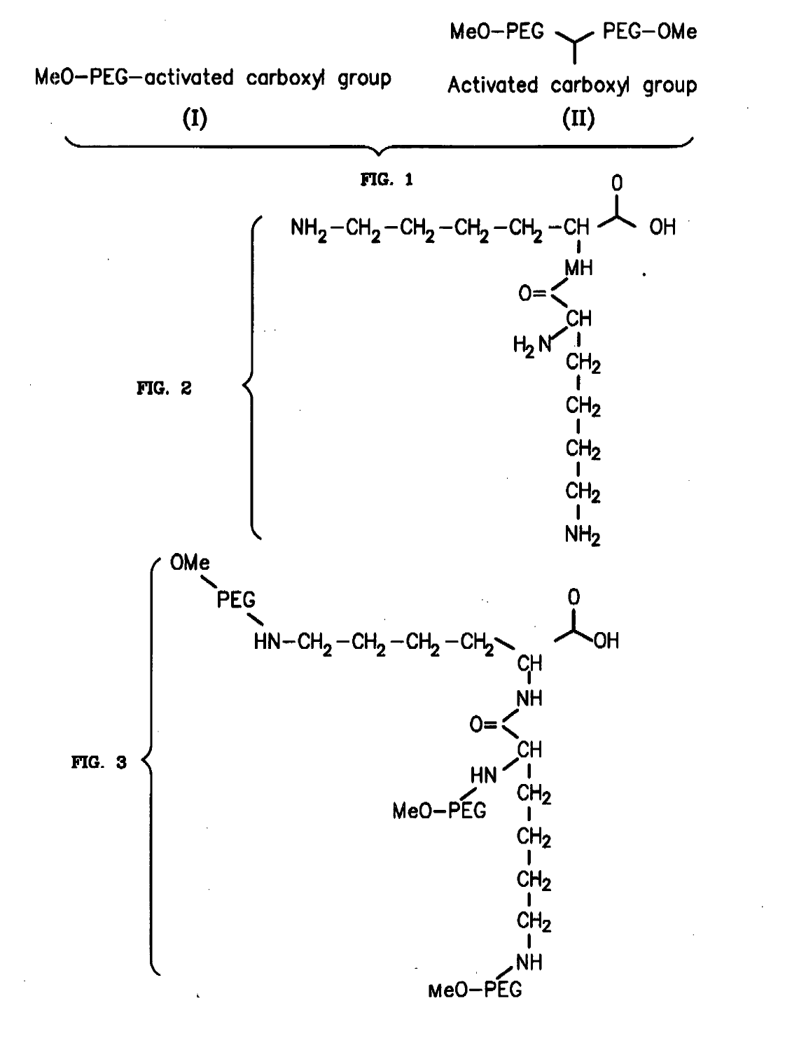

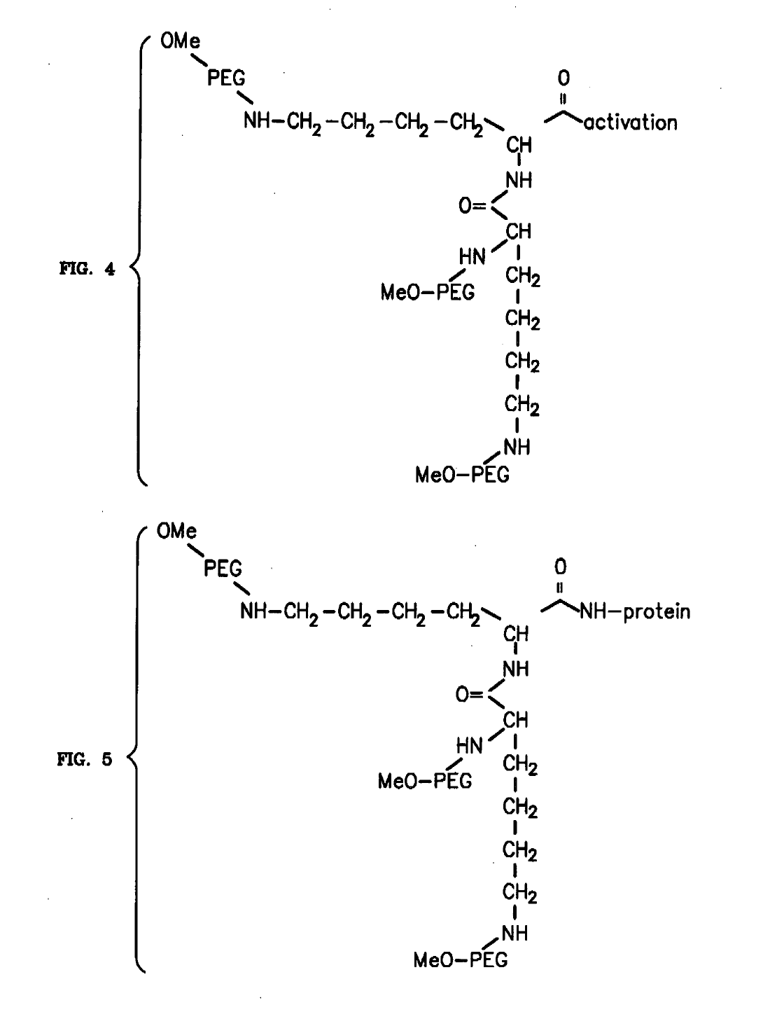

### DESCRIPTION OF THE PREFERRED EMBODIMENT

Definitions

As used herein, the term “tissue site” includes any tissues in an organism. A tissue site is typically surrounded by an aqueous or body fluid such as interstitial fluid, blood, serum, cerebrospinal fluid or peritoneal fluid.

The term “tissue defect” is a subset of “tissue site” and includes tissues, such as abraded tissue, traumatized tissue, a surgical incision or surgically resected tissue. Examples of tissue defects include, but are not limited to, surgical incisions in an internal organ such as an ovary, heart, liver, intestine, stomach, etc., wounds from injuries, surgical interventions, etc.

The term “biodegradable” means that the PEG/therapeutic agent composite will degrade over time by the action of enzymes, by hydrolytic action and/or by other similar mechanisms in the human body.

The term “bioabsorbable,” means that the PEG/therapeutic agent will be broken down and absorbed within the human body, for example, by a cell or tissue.

The term “biocompatible” means that neither the PEG/therapeutic agent nor any associated solvent will cause substantial tissue irritation or necrosis.

Proteins and peptides are an important class of therapeutics. However, when such proteins and peptides are administered therapeutically in their natural form, proteolytic enzymes can degrade them very quickly. Furthermore, such administered proteins and peptides may also elicit an immunogenic response. The short plasma half-life caused by rapid degradation requires frequent dosing, resulting in reduced compliance by patients for their needed treatments.

To address this problem, the derivatization of proteins and peptides with polyethylene glycol (PEGylation) has been established. PEGylated proteins and peptides have increased plasma half-lives and reduced immunogenicity. Traditionally a single straight-chain molecule of polyethylene glycol (PEG) is attached at a single point to a peptide or protein (shown as I in FIG. 1). Later, branched PEG (two PEGs with a single point of attachment, shown as II in FIG. 1) was developed to provide the additional steric bulk, thus providing a better polymeric shield against enzymatic degradation.

To further improve and extend the plasma half-life of desired protein or peptide therapeutics, a novel branched molecule of PEG possessing three PEGs with a single point of attachment is designed in this invention disclosure.

To further improve and extend the plasma half-life of desired protein or peptide therapeutics, a novel branched molecule of PEG possessing three PEGs with a single point of attachment (shown as III in FIG. 3) is designed in this invention disclosure.

Examples of protein or peptide therapeutics include, but not limited to, oxytocin, vasopressin, adrenocorticotrotic hormone, epidermal growth factor, platelet-derived growth factor, prolactin, luteinizing hormone releasing hormone (LHRH), LHRH agonists, LHRH antagonists, growth hormone, insulin, secretin, calcitonin, endorphins, angiotensins, tumor necrosis factor, nerve growth factor, granulocyte-colony stimulating factor, granulocyte macrophage-colony stimulating factor, glucagons-like peptide, interferon, interleukins and derivatives thereof.

Method of Manufacture

To prepare the PEG molecule (III) shown in FIG. 3, one starts with a molecule having three amine groups and one carboxyl group (seed molecule). An example of such a seed molecule is, but not limited to, dilysine. Next, the three amine groups are each coupled with an active mPEG (monomethoxy-polyethylene glycol). The active group of mPEG includes active esters, active carbonates, aldehyde, isocyanate and iminoesters. The hydrochloric salt of dilysine is dissolved in an aqueous buffer solution at pH 7.5-8.5. The resulting solution is slowly added to a pH 8.0 buffer solution containing an active methoxy-PEG (mPEG). After completing the reaction between three amine groups and the active group of each mPEG, the resulting mPEG-trisubstituted dilysine can be purified using standard purification techniques such as, but not limited to, column chromatography and precipitation methods.

1. A polyethylene glycol composition for use within a body for treatment of a condition, comprising:
a triple branched polyethylene glycol molecule; and
a biologically active substance.

2. A polyethylene glycol composition as recited in claim 1, wherein said composition is capable of inhibiting the degradation of said biologically active agent by enzymatic activity.

3. A polyethylene glycol composition as recited in claim 1, wherein said composition is capable of reducing the immunogenic properties of said biologically active agent.

4. The polyethylene glycol composition as recited in claim 1, wherein the biologically active substance comprises a therapeutic agent.

5. The polyethylene glycol composition of claim 1, wherein the bioactive substance comprises a drug.

6. The polyethylene glycol composition of claim 1, wherein the biologically active substance comprises a polypeptide.

7. The polyethylene glycol composition of claim 1, wherein the biologically active substance comprises a protein.

8. The method of manufacturing a triple branched polyethylene glycol molecule, comprising;
coupling a seed molecule having three amine groups and one carboxyl group with a monomethoxy-polyethylene glycol having an active group, said coupling forming a triple branched polyethylene glycol molecule.

9. The method of claim 8, wherein said seed molecule is dilysine.

10. The method of claim 8, wherein said active group includes esters, carbonates, aldehydes, isocyanate and iminoesters.

11. The method of manufacturing a triple branched polyethylene glycol molecule, comprising;
coupling a seed molecule having three amine groups and one carboxyl group with a monomethoxy-polyethylene glycol having an active group, said coupling forming a triple branched polyethylene glycol molecule;
activating carboxyl group and coupling activated carboxyl group with a biologically active substance.

12. The method of claim 11, wherein said seed molecule is dilysine.

13. The method of claim 11, wherein said active group includes esters, carbonates, aldehydes, isocyanate and iminoesters.

14. The method of claim 11, wherein said active substance is a peptide.

15. The method of claim 11, wherein said active substance is a protein.
